(19) United States
(12) Patent Application Publication
(10) Pub. No.: US 2008/0213181 A1
(43) Pub. Date: Sep. 4, 2008

(54) Preparation of Paramagnetic Nanoparticles Conjugated to Leukotriene B4 (LTB4) Receptor Antagonists, and Their Use as MRI Contrast Agents for the Detection of Infection and Inflammation

(75) Inventor: Thomas D. Harris, Salem, NH (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Pharma Company

(21) Appl. No.: 11/959,569

(22) Filed: Dec. 19, 2007

Related U.S. Application Data
(60) Provisional application No. 60/871,154, filed on Dec. 21, 2006.

(51) Int. Cl. A61K 49/00; C07D 401/06

(52) U.S. Cl. 424/9.1; 546/268.4; 546/256

(57) ABSTRACT

Nanoparticle-based compositions and emulsions that are specifically targeted to leukotriene B4 (LTB4) receptors, and employing LTB4 receptor antagonist targeting agents, are set forth. In addition, there is provided the use of non-antibody based compositions for such targeting. The compositions of the invention are useful as imaging agents in diagnostic and therapeutic applications.

### CROSS REFERENCE TO RELATED APPLICATION

This application claims the benefit of priority from the provisional application 60/871,154 filed Dec. 21, 2006, the contents of which are herein incorporated by reference.

### FIELD OF THE INVENTION

The invention relates to novel imaging compounds, as well as to nanoparticle-based compositions and emulsions that are specifically targeted to leukotriene B4 (LTB4) receptors, employing LTB4 receptor antagonist targeting agents. More specifically, the invention relates to non-antibody based compositions for such targeting, and to methods for their use in medical imaging.

### BACKGROUND OF THE INVENTION

Inflammation occurs in response to various forms of tissue damage. This tissue damage can result from microbial invasion, auto-immune processes, neoplasms, ischemia, tissue or organ allograft rejection, or such injurious external influences as heat, cold, radiant energy, electrical or chemical stimuli, or mechanical trauma. The ensuing inflammatory response consists of a complicated set of functional and cellular adjustments, including an increase in blood flow in the inflammatory site, an increase in capillary permeability, an efflux of fluid from the blood, and an influx and activation of inflammatory cells (leukocytes). This response pattern constitutes an important part of innate host defense mechanisms against infection, and although it carries the cost of additional tissue damage resulting from the inflammatory process itself, it ultimately promotes the subsequent repair process.

The inflammatory response cascade is initiated by a variety of soluble proinflammatory chemical substances that bind to and activate inflammatory cells (leukocytes). Leukotriene B4 (LTB4) is a potent proinflammatory lipid mediator derived from arachidonic acid via the 5-lipoxygenase pathway. It is produced by neutrophils, monocytes, macrophages, keratinocytes, lymphocytes, and mast cells. The physiological responses to LTB4 include potent neutrophil chemotactic activity, adhesion of polymorphonuclear leukosites (PMNs) to the vascular endothelium, stimulation of the release of lysosomal enzymes and superoxide radicals by PMNs, and an increase in vascular permeability. Increased levels of LTB4 have been detected in patients with asthma, acute respiratory distress syndrome, chronic obstructive pulmonary disease, contact dermatitis, cystic fibrosis, inflammatory bowel disease, gout, myocardial ischemia, psoriasis, rheumatoid arthritis, and cancer. Thus, LTB4 is an important mediator of acute and chronic inflammatory events. A number of highly potent and selective LTB4 antagonists have been reported by major pharmaceutical companies for potential therapeutic applications.

LTB4 antagonists bind to the LTB4 receptor found on leukocytes. In U.S. Pat. No. 6,416,733, it was shown that radiolabeled LTB4 antagonists are capable of imaging sites of infection and inflammation, presumably by binding to the LTB4 receptor on leukocytes found at such sites. What is now needed in the art are further novel targeting compounds using LTB4 receptor antagonist moieties, as well as new compositions of nanoparticles containing LTB4 antagonists as targeting moieties, and a method of imaging using the foregoing.

### SUMMARY OF THE INVENTION

The invention in a first embodiment is directed to novel compounds of the formula:

Wₑ—X-Lₙ-Y-L_{n′}-S_{L} (I)

In a further embodiment, there is provided a compound of the formula:

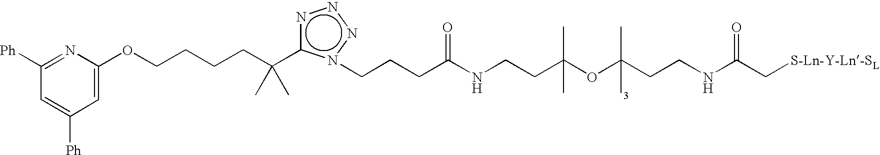

wherein Ln, Ln′, Y and S_{L} are as further defined herein. The invention is also directed to compositions and methods for imaging and drug delivery wherein non-antibody, leukotriene B4 (LTB4) receptor antagonist moieties are used as targeting agents to deliver nanoparticle emulsions to regions containing high levels of angiogenesis, such as tumors, regions of inflammation, atherosclerotic regions, and restenoses. The use of these agents in the context of imaging nanoparticle emulsions results in improved image quality, and the opportunity for targeted drug delivery.

Thus, in one aspect, the invention is directed to a method to deliver a nanoparticulate emulsion to a target tissue, wherein said target tissue is characterized as an LTB4 receptor, which method comprises administering to a subject comprising such tissue an emulsion of nanoparticles wherein said nanoparticles are coupled to a ligand specific for LTB4, with the proviso that said ligand is other than an antibody or fragment thereof.

In other aspects, the invention is directed to compositions useful in the method of the invention. The compositions have one or more targeting ligands that contain LTB4 receptor antagonists, together with one or more lipid/surfactants, that are included in a nanoparticle emulsion formulation, in which a perfluorinated hydrocarbon is used as to form the emulsion system.

Also included as part of the invention are kits containing components of the compositions that can be assembled to perform the invention methods. The kits will typically provide emulsions that contain reactive groups that can bind to targeting agents provided separately, or that can bind to ancillary substances useful for imaging or drug delivery.

### DETAILED DESCRIPTION OF THE PREFERRED EMBODIMENTS

According to the present invention, imaging of activated LTB4 receptor sites can be obtained. In one embodiment, various emulsions which are useful in imaging can be employed. When used alone, the nanoparticle-containing emulsions are useful as contrast agents for ultrasound imaging. For use in magnetic resonance imaging or in X-ray imaging, it may be desirable to employ a transition metal as a contrast agent; if the nanoparticles comprise fluorocarbons, however, the fluorocarbon itself is useful in obtaining an image. Radionuclides are also useful both as diagnostic and therapeutic agents. In addition, reagents for optical imaging, such as fluorophores may also be associated with the nanoparticles. In addition, or alternatively, the nanoparticles in the emulsion may contain one or more bioactive agents.

Any nanoparticulate emulsion may be used. For example, PCT publication WO95/03829 describes oil emulsions where the drug is dispersed or solubilized inside an oil droplet and the oil droplet is targeted to a specific location by means of a ligand. U.S. Pat. No. 5,542,935 describes site-specific drug delivery using gas-filled perfluorocarbon microspheres. The drug delivery is accomplished by permitting the microspheres to home to the target and then effecting their rupture. Low boiling perfluoro compounds are used to form the particles so that the gas bubbles can form.

However, it is preferred to employ emulsions wherein the nanoparticles are based on high boiling perfluorocarbon liquids such as those described in U.S. Pat. No. 5,958,371 referenced above. The liquid emulsion contains nanoparticles comprised of relatively high boiling perfluorocarbons surrounded by a coating which is composed of a lipid and/or surfactant. The surrounding coating is able to couple directly to a targeting moiety or can entrap an intermediate component which is covalently coupled to the targeting moiety, optionally through a linker, or may contain a non-specific coupling agent such as biotin. Alternatively, the coating may be cationic so that negatively charged targeting agents such as nucleic acids, in general, or aptamers, in particular, can be adsorbed to the surface.

In addition to the targeting LTB4 ligand, the nanoparticles may contain associated with their surface an “ancillary agent” useful in imaging and/or therapy a radionuclide, a contrast agent for magnetic resonance imaging (MRI) or for X-ray imaging, a fluorophore and/or a biologically active compound. The nanoparticles themselves can serve as contrast agents for ultrasound imaging.

The preferred emulsion is a nanoparticulate system containing a high boiling perfluorocarbon as a core and an outer coating that is a lipid/surfactant mixture which provides a vehicle for binding a multiplicity of copies of one or more desired components to the nanoparticle. The construction of the basic particles and the formation of emulsions containing them, regardless of the components bound to the outer surface is described in the above-cited patents, U.S. Pat. Nos. 5,690,907 and 5,780,010; and patents issued on daughter applications U.S. Pat. Nos. 5,989,520 and 5,958,371; and incorporated herein by reference.

The high boiling fluorochemical liquid is such that the boiling point is higher than that of body temperature—i.e., 37 degree C. Thus, fluorochemical liquids which have boiling points at least 30 degree C. are preferred, more preferably 37 degree C., more preferably above 50 degree C., and most preferably above about 90 degree C. The “fluorochemical liquids” useful in the invention include straight and branched chain and cyclic perfluorocarbons including perfluorinated compounds which have other functional groups. “Perfluorinated compounds” includes compounds that are not pure perfluorocarbons but rather wherein other halogen groups may be present. These include perfluorooctylbromide, and perfluorodichlorooctane, for example.

Perfluorinated compounds as thus defined are preferred.

Useful perfluorocarbon emulsions are disclosed in U.S. Pat. Nos. 4,927,623, 5,077,036, 5,114,703, 5,171,755, 5,304,325, 5,350,571, 5,393,524, and 5,403,575, which are incorporated herein by reference, and include those in which the perfluorocarbon compound is perfluorodecalin, perfluorooctane, perfluorodichlorooctane, perfluoro-n-octyl bromide, perfluoroheptane, perfluorodecane, perfluorocyclohexane, perfluoromorpholine, perfluorotripropylamine, perfluortributylamine, perfluorodimethylcyclohexane, perfluorotrimethylcyclohexane, perfluorodicyclohexyl ether, perfluoro-n-butyltetrahydrofuran, and compounds that are structurally similar to these compounds and are partially or fully halogenated (including at least some fluorine substituents) or partially or fully fluorinated including perfluoroalkylated ether, polyether or crown ether.

The lipid/surfactants used to form an outer coating on the nanoparticles (that will contain the coupled ligand or entrap reagents for binding desired components to the surface) include natural or synthetic phospholipids, fatty acids, cholesterols, lysolipids, sphingomyelins, and the like, including lipid conjugated polyethylene glycol. Various commercial anionic, cationic, and nonionic surfactants can also be employed, including Tweens, Spans, Tritons, and the like, such as sodium dodecylsulfate (SDS). Some surfactants are themselves fluorinated, such as perfluorinated alkanoic acids such as perfluorohexanoic and perfluorooctanoic acids, perfluorinated alkyl sulfonamide, alkylene quaternary ammonium salts and the like. In addition, perfluorinated alcohol phosphate esters can be employed. Cationic lipids included in the outer layer may be advantageous in entrapping ligands such as nucleic acids, in particular aptamers. Typical cationic lipids may include DOTMA, N-[1-(2,3-dioleoyloxy)propyl]-N,N,N-trimethylammonium chloride; DOTAP, 1,2-dioleoyloxy-3-(trimethylammon-io)propane; DOTB, 1,2-dioleoyl-3-(4′-trimethylammonio)butanoyl-sn-glycerol-, 1,2-diacyl-3-trimethylammonium-propane; 1,2-diacyl-3-dimethylammonium-pr-opane; 1,2-diacyl-sn-glycerol-3-ethyl phosphocholine; and 3.beta.-[N′,N′-dimethylaminoethane)-carbamol]cholesterol-HCl. Other important lipids include the phospholipids, and particularly preferred among these are DPPA (dipalmitoylphosphatidic acid), DPPC (dipalmitoylphosphatidylcholine), DPPE (dipalmitoylphosphatidylethanolamine), dialkylphosphatidyl ethanolamine, dialkylphosphatidyl choline, and dialkylphosphatidyl serine.

In preferred embodiments, included in the lipid/surfactant coating are components with reactive groups that can be used to couple the LTB4 receptor antagonist ligand and/or the ancillary substance useful for imaging or therapy. As will be described below, the lipid/surfactant components can be coupled to these reactive groups through functionalities contained in the lipid/surfactant component. For example, phosphatidylethanolamine may be coupled through its amino group directly to a desired moiety, or may be coupled to a linker such as a short peptide which may provide carboxyl, amino, or sulfhydryl groups as described below. Alternatively, standard linking agents such a maleimides may be used. A variety of methods may be used to associate the targeting ligand and the ancillary substances to the nanoparticles; these strategies may include the use of spacer groups such as polyethyleneglycol or peptides, for example.

The lipid/surfactant coated nanoparticles are typically formed by microfluidizing a mixture of the fluorocarbon lipid which forms the core and the lipid/surfactant mixture which forms the outer layer in suspension in aqueous medium to form an emulsion. In this procedure, the lipid/surfactants may already be coupled to additional ligands when they are coated onto the nanoparticles, or may simply contain reactive groups for subsequent coupling. Alternatively, the components to be included in the lipid/surfactant layer may simply be solubilized in the layer by virtue of the solubility characteristics of the ancillary material. Sonication or other techniques may be required to obtain a suspension of the lipid/surfactant in the aqueous medium. Typically, at least one of the materials in the lipid/surfactant outer layer comprises a linker or functional group which is useful to bind the additional desired component or the component may already be coupled to the material at the time the emulsion is prepared.

For coupling by covalently binding the targeting ligand or other organic moiety (such as a chelating agent for a paramagnetic metal) to the components of the outer layer, various types of bonds and linking agents may be employed. Typical methods for forming such coupling include formation of amides with the use of carbodiimides, or formation of sulfide linkages through the use of unsaturated components such as maleimide. Other coupling agents include, for example, glutaraldehyde, propanedial or butanedial, 2-iminothio lane hydrochloride, bifunctional N-hydroxysuccinimide esters such as disuccinimidyl suberate, disuccinimidyl tartrate, bis[2-(succinimidooxycarbonyloxy)ethyl]sulfone, heterobifunctional reagents such as N-(5-azido-2-nitrobenzoyloxy)succinimide, succinimidyl 4-(N-maleimidomethyl)cyclohexane-1-carboxylate, and succinimidyl 4-(p-maleimidophenyl)butyrate, homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like. Linkage can also be accomplished by acylation, sulfonation, reductive amination, and the like. A multiplicity of ways to couple, covalently, a desired ligand to one or more components of the outer layer is well known in the art. The ligand itself may be included in the surfactant layer if its properties are suitable. For example, if the ligand contains a highly lipophilic portion, it may itself be embedded or partially embedded in the lipid/surfactant coating. Further, if the ligand is capable of direct adsorption to the coating, this too will effect its coupling. For example, nucleic acids, because of their negative charge, adsorb directly to cationic surfactants.

The ligand may bind directly to the nanoparticle, i.e., the ligand is associated with the nanoparticle itself. Alternatively, indirect binding such as that effected through biotin/avidin may be employed typically for the LTB4-specific ligand. For example, in biotin/avidin mediated targeting, the LTB4 receptor antagonist ligand is coupled not to the emulsion, but rather coupled, in biotinylated form to the targeted tissue.

Ancillary agents that may be coupled to the nanoparticles through entrapment in the coating layer include radionuclides. Radionuclides may be either therapeutic or diagnostic; diagnostic imaging using such nuclides is well known and by targeting radionuclides to undesired tissue a therapeutic benefit may be realized as well. Typical diagnostic radionuclides include ¹⁸F, ^{99m}Tc, ⁹⁵Tc, ¹¹¹In, ⁶²Cu, ⁶⁴Cu, ⁶⁷Ga, and ⁶⁸Ga, and therapeutic nuclides include ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, 169Yb, ¹⁷⁵Yb, ¹⁶⁵Dy, ¹⁶⁶Dy, ⁶⁷Cu, ¹⁰⁵Rh, ¹¹¹Ag, and ¹⁹²Ir. The nuclide can be provided to a preformed emulsion in a variety of ways. For example, ^{99m}Tc-pertechnate may be mixed with an excess of stannous chloride and incorporated into the preformed emulsion of nanoparticles. Stannous oxinate can be substituted for stannous chloride. In addition, commercially available kits, such as the HM-PAO (exametazine) kit marketed as Ceretek® by Nycomed Amersham can be used. Means to attach various radioligands to the nanoparticles of the invention are understood in the art.

Chelating agents containing paramagnetic metals for use in magnetic resonance imaging can also be employed as ancillary agents. Typically, a chelating agent containing a paramagnetic metal is associated with the lipids/surfactants of the coating on the nanoparticles and incorporated into the initial mixture which is sonicated. The chelating agent can be coupled directly to one or more of components of the coating layer. Suitable chelating agents include a variety of multi-dentate compounds including EDTA, DPTA, DOTA, and the like. Most preferred embodiments include macrocyclic polyamines such as cyclen which have been functionalized with one or more alkyl carboxylic acid groups. These chelating agents can be coupled directly to functional groups contained in, for example, phosphatidyl ethanolamine, bis-oleate, and the like, or through linking groups.

The paramagnetic metals useful in the MRI contrast agents of the invention include rare earth metals, typically, manganese, ytterbium, gadolinium, europium, and the like. Iron ions may also be used.

Other ancillary agents include fluorophores such as fluorescein, 5-dimethylamino-1-naphthylsulfonyl(dansyl), quantum dots, and the like.

Included in the surface of the nanoparticle, in some embodiments of the invention, are biologically active agents. These biologically active agents can be of a wide variety, including proteins, nucleic acids, pharmaceuticals, and the like. Thus, included among suitable pharmaceuticals are antineoplastic agents, hormones, analgesics, anesthetics, neuromuscular blockers, antimicrobials or antiparasitic agents, antiviral agents, interferons, antidiabetics, antihistamines, antitussives, anticoagulants, and the like.

In all of the foregoing cases, whether the associated moiety is a targeting ligand for LTB4 or is an ancillary agent, the defined moiety may be non-covalently associated with the lipid/surfactant layer, may be directly coupled to the components of the lipid/surfactant layer, or may be coupled to said components through spacer moieties.

Targeting Ligands

The emulsions and nanoparticles of the present invention utilize targeting agents that are ligands specific for LTB4 receptors, other than an antibody or fragment thereof Such compounds derivatized with appropriate imaging agents have potential use as diagnostic agents. In particular, these compounds are set forth and described in U.S. Pat. No. 6,416,733, incorporated herein by reference. The targeting compounds are represented by the general formulas: Wₑ—X-Lₙ-Y-L_{n′}-S_{L}; Wₑ-X-Lₙ(L_{n′}-S_{L})—Y; or Z-L_{n′}-S_{L}, as set forth in the '733 patent, with the exception that in place of Cₕ, there is provided a lipid/surfactant S_{L} as hereinabove described for binding the LTB4 moiety to the nanoparticle. In the above formulas, Lₙ is a linking group having the formula

(CR⁸R⁹)_{g}—(W¹)ₕ-(M¹)ₖ-(CR¹⁰R¹¹)_{g}

wherein,

R⁸, R⁹, R¹⁰ and R¹¹ are independently selected at each occurrence from the group: a bond to L_{n′}, H, and C₁-C₃ alkyl or R¹⁰ and R¹¹ may be taken together to form a 3-6 membered cycloalkyl or heterocycle;

W¹ is O;

M¹ is selected from the group of: phenyl substituted with 0-1 R¹², heterocycle substituted with 0-1 R¹², benzophenone substituted with 0-1 R¹², and diphenylether substituted with 0-1 R¹²;

R¹² is independently selected from the group: a bond to L_{n′}, —COOR¹³, C₁-C₅ alkyl substituted with 0-1 R¹⁴, and C₁-C₅ alkoxy substituted with 0-1 R¹⁴;

R¹³ is H or C₁-C₅ alkyl:

R¹⁴ is independently selected from the group: a bond to L_{n′}, and —COOH;

g is 0-10;

h is 0-3;

k is 0-1;

g is 0-5; provided that when h is 0 and k is 0, g is >1; and provided that when W¹ is O or S and k is 0, g+g′ is ≧1;

Y is selected from C(═O)NH, NHC(═O), C═O, C(═O)O, OC(═O), NHS(═O)₂, C(═O)NHS(═O)₂, COOH, C(═O)NH₂, NH(C═O)NH, S, or tetrazole;

provided that from 0-1 of R⁹, R¹⁰, R¹¹, R¹², and R¹⁴ is a bond to L_{n′} and when one of these variables is a bond to L_{n′} then Y is COOH, C(═O)NH₂, or tetrazole;

L_{n′} is a linking group having the formula:

(W²)_{h′}—(CR¹⁵R¹⁶)_{g″}-(M²)_{k′}-(W²)_{h″}—(CR¹⁷R¹⁸)_{g′″}—(W²)_{h′″}

wherein, W² is independently selected at each occurrence from the group: O, S, NH, NHC(═O), C(═O)NH, C(═O), C(═O)O, OC(═O), NHC(═O)NH, SO₂, (OCH₂CH₂)ₛ, (CH₂CH₂O)_{s′}, (OCH₂CH₂CH₂)_{s″}, (CH₂CH₂CH₂O)ₜ, CH₂S, SCH₂, and (aa)_{t′}, wherein aa is independently at each occurrence an amino acid, and s, s′, s″, t, and t′ are independently 1-10;

M² is selected from the group of: aryl substituted with 0-1 R¹⁹, cycloalkyl substituted with 0-3 R¹⁹, and heterocycle substituted with 0-1 R¹⁹;

R¹⁵, R¹⁶, R¹⁷ and R¹⁸ are independently selected at each occurrence from the group: ═O, COOH, SO₃H, PO₃H, C₁-C₅ alkyl substituted with 0-3 R¹⁹, aryl substituted with 0-3 R¹⁹, benzyl substituted with 0-3 R¹⁹, and C₁-C₅ alkoxy substituted with 0-3 R¹⁹, NHC(═O)R²⁰ , C(═O)NHR²⁰, NHC(═O)NHR²⁰, NHR²⁰, R²⁰, and a bond to a lipid/surfactant;

Optionally, R¹⁷ and R¹⁸ may form a 4-7 membered heterocyclic or aliphatic ring;

R¹⁹ is independently selected at each occurrence from the group of: COOR²⁰, OH, NHR²⁰, SO₃H, PO₃H, aryl substituted with 0-3 R²⁰, heterocycle substituted with 0-3 R²⁰, C₁-C₅ alkyl substituted with 0-1 R²¹, C₁-C₅ alkoxy substituted with 0-1 R²¹, a bond, and a bond to a lipid/surfactant;

R²⁰ is independently selected at each occurrence from the group of: H, aryl substituted with 0-1 R²¹, heterocycle substituted with 0-1 R²¹, cycloalkyl substituted with 0-1 R²¹, polyalkylene glycol substituted with 0-1 R²¹, carbohydrate substituted with 0-1 R²¹, cyclodextrin substituted with 0-1 R²¹, amino acid substituted with 0-1 R²¹, polycarboxyalkyl substituted with 0-1 R²¹, polyazaalkyl substituted with 0-1 R²¹, peptide substituted with 0-1 R²¹, wherein said peptide is comprised of 2-10 amino acids, and a bond to a lipid/surfactant;

R²¹ is a bond to a lipid/surfactant;

and k′ is 0-2; h′ is 0-2; h″ is 0-5; h′″ is 0-2; g″ is 0-10; g′″ is 0-10.

Particularly preferred targeting ligands include 2-alkoxy-4-arly-6-aryl′-pyridines in which the alkoxy group may or may not contain a 5-tetrazole which in turn may be attached to Lₙ per the above formula. The alkoxy group may have a total chain length or between three and twenty atoms, with the chain length preferably between five and ten, most preferably six. The aryl and aryl′ groups may be the same or different and are preferably mono- or bi-cyclic aromatic hydrocarbons or heterocycles. In formula I, the group Y may be represented by a succinimide (derived from a maleimide), the linking group Lₙ by a gamma amino acid.

In one embodiment of the invention, a particularly preferred compound is referred to as SJ669, which has been reported by Rhone-Poulenc Rorer as a potential anti-inflammatory agent. SJ669 binds with high affinity to the LTB4 receptor. It has been discovered that Hynic (a high affinity ligand for Tc-99m) can be tethered to SJ669 at the tetrazole ring to give SG380 below with no loss of affinity for the LTB4 receptor.

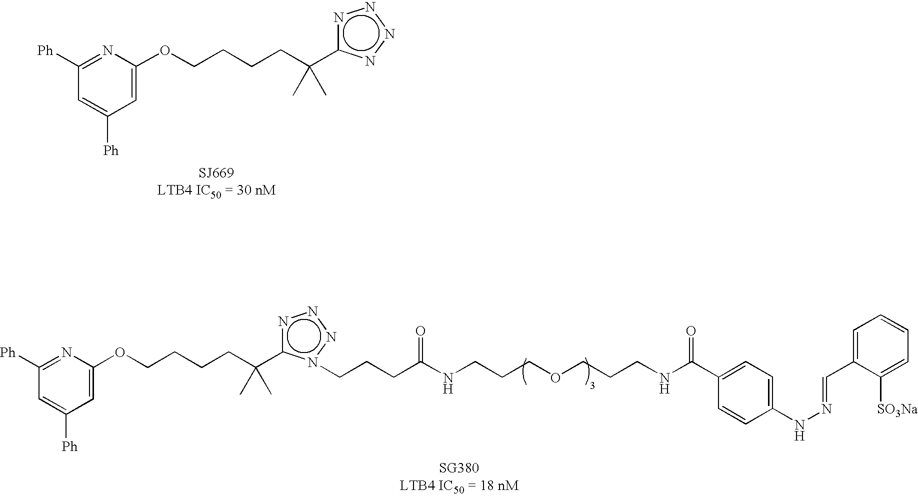

By one method the amine precursor of SG380 (1) can then be coupled to trityl-protected thiolacetic acid, followed by deprotection to give free thiol 3.

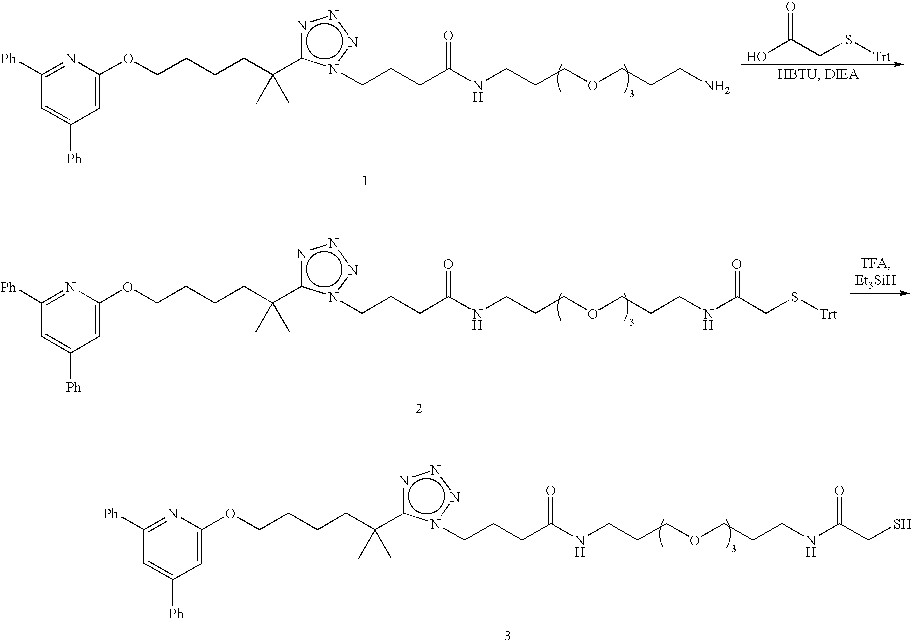

Free thiol 3 can be conjugated to nanoparticles that are derivatized with either the maleimide or α-haloacetyl group. Alternatively, 3 could first be conjugated with a lipid/surfactant, such as a phospholipid, in solution via either a maleimide or α-haloacetyl group, and the conjugate could then be incorporated into the surface of a lipophilic nanoparticle.

The LTB4 moiety of the invention can incorporate any of the structural modifications as described in U.S. Pat. No. 6,416,733. This includes classes of compounds other than the diphenylpyridyl compounds of which SG380 is a member. In addition to the thiol chemistry described above, immobilization to nanoparticles can take many forms. For example, LTB4 antagonist having free amines can be conjugated to nanoparticles derivatized with reactive carboxyl group derivatives such as active esters, acid halides, or imidoesters. Alternatively, amines can be coupled to aldehydes or ketones by reductive amination procedures.

Thus, the targeting ligand and lipid/surfactant combination may be represented by the preferred formula of Formula I, wherein S_{L} represents the lipid/surfactant moieties previously described herein:

Wₑ—X-Lₙ-Y-L_{n′}-S_{L} (I)

Particularly preferred is the compound of the formula set forth below:

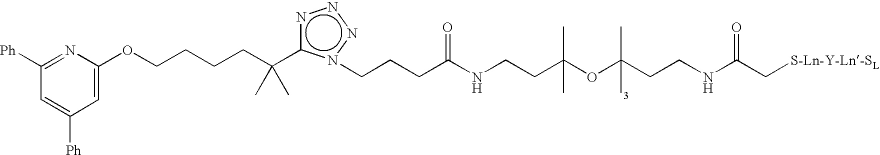

wherein S_{L} is a lipid/surfactant as set forth above. More preferably, S_{L} represents at least one of the preferred phospholipids from the group of DPPA, DPPC, DPPE, dialkylphosphatidyl ethanolamine, dialkylphosphatidyl choline, and dialkylphosphatidyl serine. Alternatively, S_{L} can also represent or a cationic lipid selected from the group consisting of DOTMA, N-[1-(2,3-dioleoyloxy)propyl]-N,N,N-trimethylammonium chloride; DOTAP, 1,2-dioleoyloxy-3-(trimethylammon-io)propane; DOTB, 1,2-dioleoyl-3-(4′-trimethylammonio)butanoyl-sn-glycerol-, 1,2-diacyl-3-trimethylammonium-propane; 1,2-diacyl-3-dimethylammonium-pr-opane; 1,2-diacyl-sn-glycerol-3-ethyl phosphocholine; and 3.beta.-[N′,N′-dimethylaminoethane)-carbamol]cholesterol-HCl.

Preparation Methods

In a typical procedure for preparing the emulsions of the invention, the fluorochemical liquid and the components of the lipid/surfactant coating are fluidized in aqueous medium to form an emulsion. The functional components of the surface layer may be included in the original emulsion, or may later be covalently coupled to the surface layer subsequent to the formation of the nanoparticle emulsion. In one particular instance, for example, where a nucleic acid targeting agent or drug is to be included, the coating may employ a cationic surfactant and the nucleic acid adsorbed to the surface after the particle is formed.

When appropriately prepared, the nanoparticles that comprise ancillary agents contain a multiplicity of functional such agents at their outer surface, the nanoparticles typically contain hundreds or thousands of molecules of the biologically active agent, targeting ligand, radionuclide and/or MRI contrast agent. For MRI contrast agents, the number of copies of a component to be coupled to the nanoparticle is typically in excess of 5,000 copies per particle, more preferably 10,000 copies per particle, still more preferably 30,000, and still more preferably 50,000-100,000 or more copies per particle. The number of targeting agents per particle is typically less, of the order of several hundred while the concentration of fluorophores, radionuclides, and biologically active agents is also variable.

The nanoparticles need not contain an ancillary reporting agent. In general, the targeted particles, directly coupled to a LTB4 receptor antagonist ligand, are useful themselves as ultrasound contrast agents. Further, because the particles have a fluorocarbon core, ¹⁹F magnetic resonance imaging can be used to track the location of the particles concomitantly with their additional functions described above. However, the inclusion of other components in multiple copies renders them useful in other respects. For instance, the inclusion of a chelating agent containing a paramagnetic ion makes the emulsion useful as a magnetic resonance imaging contrast agent. The inclusion of biologically active materials makes them useful as drug delivery systems. The inclusion of radionuclides makes them useful either as therapeutic for radiation treatment or as diagnostics for imaging. Other imaging agents include fluorophores, such as fluorescein or dansyl. Biologically active agents may be included. A multiplicity of such activities may be included; thus, images can be obtained of targeted tissues at the same time active substances are delivered to them.

The emulsions can be prepared in a range of methods depending on the nature of the components to be included in the coating. In a typical procedure, used for illustrative purposes only, the following procedure is set forth: Perfluorooctylbromide (40% w/v, PFOB, 3M), and a surfactant co-mixture (2.0%, w/v) and glycerin (1.7%, w/v) is prepared where the surfactant co-mixture includes 64 mole % lecithin (Pharmacia Inc), 35 mole % cholesterol (Sigma Chemical Co.) and 1 mole % dipalmitoyl-L-alpha-phosphatidyl-ethanolamine, Pierce Inc.) dissolved in chloroform. A drug is suspended in methanol (ca. 25 μg/20 μl) and added in titrated amounts between 0.01 and 5.0 mole % of the 2% surfactant layer, preferably between 0.2 and 2.0 mole %. The chloroform-lipid mixture is evaporated under reduced pressure, dried in a 50 degree C. vacuum oven overnight and dispersed into water by sonication. The suspension is transferred into a blender cup (Dynamics Corporation of America) with perfluorooctylbromide in distilled or deionized water and emulsified for 30 to 60 seconds. The emulsified mixture is transferred to a Microfluidics emulsifier (Microfluidics Co.) and continuously processed at 20,000 PSI for three minutes. The completed emulsion is vialed, blanketed with nitrogen and sealed with stopper crimp seal until use. A control emulsion can be prepared identically excluding the drug from the surfactant commixture. Particle sizes are determined in triplicate at 37° C. with a laser light scattering submicron particle size analyzer (Malvern Zetasizer 4, Malvern Instruments Ltd., Southborough, Mass.), which indicate tight and highly reproducible size distribution with average diameters less than 400 nm. Unincorporated drug can be removed by dialysis or ultrafiltration techniques. To provide the targeting ligand, an LTB4 receptor antagonist ligand is coupled covalently to the phosphatidyl ethanolamine through a bifunctional linker in the procedure described above. Other procedures for preparing the emulsions herein described may be utilized by the skilled artisan.

Kits

The emulsions of the invention may be prepared and used directly in the methods of the invention, or the components of the emulsions may be supplied in the form of kits. The kits may comprise the pre-prepared targeted composition containing all of the desired ancillary materials in buffer or in lyophilized form. Alternatively, the kits may include a form of the emulsion which lacks the LTB4 receptor antagonist ligand which is supplied separately. Under these circumstances, typically, the emulsion will contain a reactive group, such as a maleimide group, which, when the emulsion is mixed with the targeting agent, effects the binding of the targeting agent to the emulsion itself. A separate container may also provide additional reagents useful in effecting the coupling. Alternatively, the emulsion may contain reactive groups which bind to linkers coupled to the desired component to be supplied separately which itself contains a reactive group. A wide variety of approaches to constructing an appropriate kit may be envisioned. Individual components which make up the ultimate emulsion may thus be supplied in separate containers, or the kit may simply contain reagents for combination with other materials which are provided separately from the kit itself.

Applications

The emulsions and kits for their preparation are useful in the methods of the invention which include imaging of tissues containing high expression levels of LTB4, and where tissues with such expression levels are undesirable, treatment. High expression levels of LTB4 are typical of activated endothelial cells and are considered diagnostic for inflammation.

The diagnostic radiopharmaceuticals are administered by intravenous injection, usually in saline solution, at a dose of 1 to 100 mCi per 70 kg body weight, or preferably at a dose of 5 to 50 mCi. Imaging is performed using known procedures.

The therapeutic radiopharmaceuticals are administered by intravenous injection, usually in saline solution, at a dose of 0.01 to 5 mCi per kg body weight, or preferably at a dose of 0.1 to 4 mCi per kg body weight. For comparable therapeutic radiopharmaceuticals, current clinical practice sets dosage ranges from 0.3 to 0.4 mCi/kg for Zevalin™ to 1-2 mCi/kg for OctreoTher™, a labeled somatostatin peptide. For such therapeutic radiopharmaceuticals, there is a balance between tumor cell kill vs. normal organ toxicity, especially radiation nephritis. At these levels, the balance generally favors the tumor cell effect. These dosages are higher than corresponding imaging isotopes.

The magnetic resonance imaging contrast agents of the present invention may be used in a similar manner as other MRI agents as described in U.S. Pat. No. 5,155,215; U.S. Pat. No. 5,087,440; Margerstadt, et al., Magn. Reson. Med. (1986) 3:808; Runge, et al., Radiology (1988) 166:835; and Bousquet, et al., Radiology (1988) 166:693. Other agents that may be employed are those set forth in U.S. Pat. No. 2002/0,127,182 which are pH sensitive and can change the contrast properties dependent on pulse. Generally, sterile aqueous solutions of the contrast agents are administered to a patient intravenously in dosages ranging from 0.01 to 1.0 mmoles per kg body weight.

A particularly preferred set of MRI chelating agents includes 1,4,7,10-tetraazacyclododecane-1,4,7,10-tetraacetic acid (DOTA) and its derivatives, in particular, a methoxybenzyl derivative comprising an isothiocyanate functional group (DOTA-NCS) which can then be coupled to the amino group of a phosphatidyl ethanolamine (PE) or to a peptide derivatized form thereof. Derivatives of this type are described in U.S. Pat. No. 5,573,752, incorporated herein by reference. Other suitable chelating agents are disclosed in U.S. Pat. No. 6,056,939, also incorporated herein by reference.

The DOTA isocyanate derivative can also be coupled to the lipid/surfactant directly or through a peptide spacer. The use of gly-gly-gly as a spacer is illustrated in the reaction scheme below. For direct coupling, the DOTA-isocyanate is simply reacted with PE to obtain the coupled product. When a peptide is employed, for example a triglycyl link, phosphatidyl ethanolamine (PE) is first coupled to t-boc protected triglycine. Standard coupling techniques, such as forming the activated ester of the free acid of the t-boc-triglycine using diisopropyl carbodiimide (or a functional equivalent thereof) with either N-hydroxy succinimide (NHS) or hydroxybenzotriazole (HBT) are employed and the resultant t-boc-triglycine-PE is purified.

Treatment of the t-boc-triglycine-PE with trifluoroacetic acid yields triglycine-PE, which is then reacted with excess DOTA-NCS in DMF/CHCl₃ at 50° C. The final product is isolated by removing the solvent, followed by rinsing the remaining solid with excess water, to remove excess solvent and any un-reacted or hydrolyzed DOTA-NCS.

For use as X-ray contrast agents, the compositions of the present invention should generally have a heavy atom concentration of 1 mM to 5 M, preferably 0.1 M to 2 M. Dosages, administered by intravenous injection, will typically range from 0.5 mmol/kg to 1.5 mmol/kg, preferably 0.8 mmol/kg to 1.2 mmol/kg. Imaging is performed using known techniques, preferably X-ray computed tomography.

The ultrasound contrast agents of the present invention are administered by intravenous injection in an amount of 10 to 30 μL of the echogenic gas per kg body weight or by infusion at a rate of approximately 3 μL/kg/min. Imaging is performed using known techniques of sonography.

The methods of employing the nanoparticulate emulsions of the invention are well known to those in the art. Typically, the tissues of interest to be imaged or treated include areas of inflammation, which may characterize a variety of disorders including rheumatoid arthritis, areas of irritation such as those affected by angioplasty resulting in restenosis, tumors, and areas affected by atherosclerosis.

A non-exhaustive list of combinations might include: emulsion preparations that contain, in their lipid-surfactant layer, an ancillary component such as a fluorophore or chelating agent and reactive moieties for coupling to the LTB4 targeting agent; the converse where the emulsion is coupled to targeting agent and contains reactive groups for coupling to an ancillary material; emulsions which contain both targeting agent and a chelating agent but wherein the metal to be chelated is either supplied in the kit or independently provided by the user; preparations of the nanoparticles comprising the surfactant/lipid layer where the materials in the lipid layer contain different reactive groups, one set of reactive groups for a LTB4 receptor antagonist ligand and another set of reactive groups for an ancillary agent; preparation of emulsions containing any of the foregoing combinations where the reactive groups are supplied by a linking agent.

The following examples illustrate various preferred embodiments of the invention, but should not be construed as limiting the scope thereof:

### EXAMPLE 1

### Synthesis of (2R,5R,8R,11R)-32-(5-(6-(4,6-Diphenylpyridin-2-yloxy)-2-methylhexan-2-yl)-1H-tetrazol-1-yl)-2-(2-mercaptoacetamido)-3,6,9,12,28-pentaoxo-5,8,11-tris(sulfomethyl)-17,20,23-trioxa-4,7,10,13,27-pentaazadotriacontane-1-sulfonic Acid

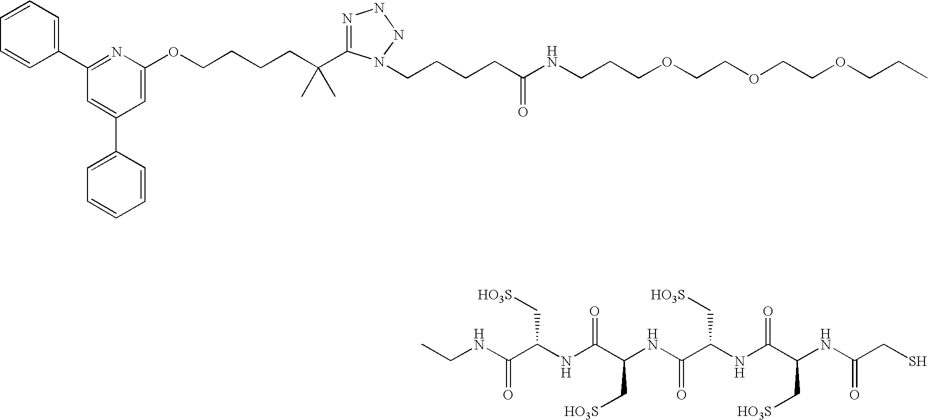

### Part A—Preparation of (2R,5R,8R,11R)-32-(5-(6-(4,6-Diphenylpyridin-2-yloxy)-2-methylhexan-2-yl)-1H-tetrazol-1-yl)-3,6,9,12,28-pentaoxo-5,8,11-tris(sulfomethyl)-2-(2-(tritylthio)acetamido)-17,20,23-trioxa-4,7,10,13,27-pentaazadotriacontane-1-sulfonic Acid

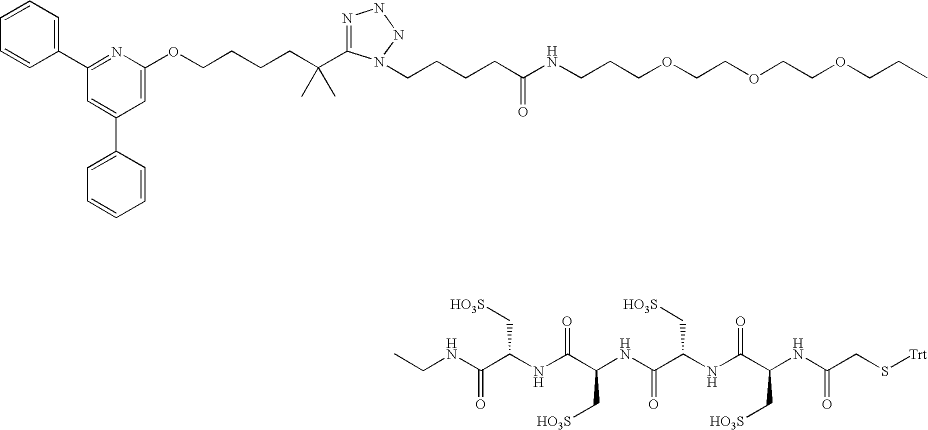

A solution of 2-((triphenylmethyl)thio)acetic acid (Brenner, D., et al. Inorg. Chem. 1984, 23, 3793-3797) and HOAt in DMF is pre-activated by the addition of HBTU and sufficient DIEA to maintain pH 8-9. To the solution is added (2R,5R,8R,11R)-2-amino-32-(5-(6-(4,6-diphenylpyridin-2-yloxy)-2-methylhexen-2-yl)-1H-tetrazol-1-yl)-3,6,9,12,28-pentaoxo-5,8,11-tris(sulfomethyl)-17,20,23-trioxa-4,7,10,13,27-pentaazadotriacontane-1-sulfonic acid (Harris, T. D., et al. J. Med. Chem. 2005, 48, 6442-6253), and the solution is stirred at room temperature under nitrogen for 18 h. DMF is removed in vacuo and the crude product is purified by preparative reverse phase HPLC to give the title compound.

### Part B—Preparation of (2R,5R,8R,11R)-32-(5-(6-(4,6-Diphenylpyridin-2-yloxy)-2-methylhexan-2-yl)-1H-tetrazol-1-yl)-2-(2-mercaptoacetamido)-3,6,9,12,28-pentaoxo-5,8,11-tris(sulfomethyl)-17,20,23-trioxa-4,7,10,13,27-pentaazadotriacontane-1-sulfonic Acid

A solution of the product of Part A in TFA:triethylsilane (95:5) is heated at 70° C. under nitrogen for 1 h. The solution is concentrated on a rotary evaporator and dried further under high vacuum. The resulting residue is purified by reverse phase HPLC to give the title compound.

### EXAMPLE 2

### Synthesis of (2R,5R,8R,11R,14S,19R,22R,25R,28R)-2,28-bis(19-(5-(6-(4,6-Diphenylpyridin-2-yloxy)-2-methylhexan-2-yl)-1H-tetrazol-1-yl)-15-oxo-4,7,10-trioxa-14-azanonadecylcarbamoyl)-14-(2-mercaptoacetamido)-4,7,10,13,17,20,23,26-octaoxo-5,8,11,19,22,25-hexakis(sulfomethyl)-3,6,9,12,18,21,24,27-octaazanonacosane-1,29-disulfonic Acid

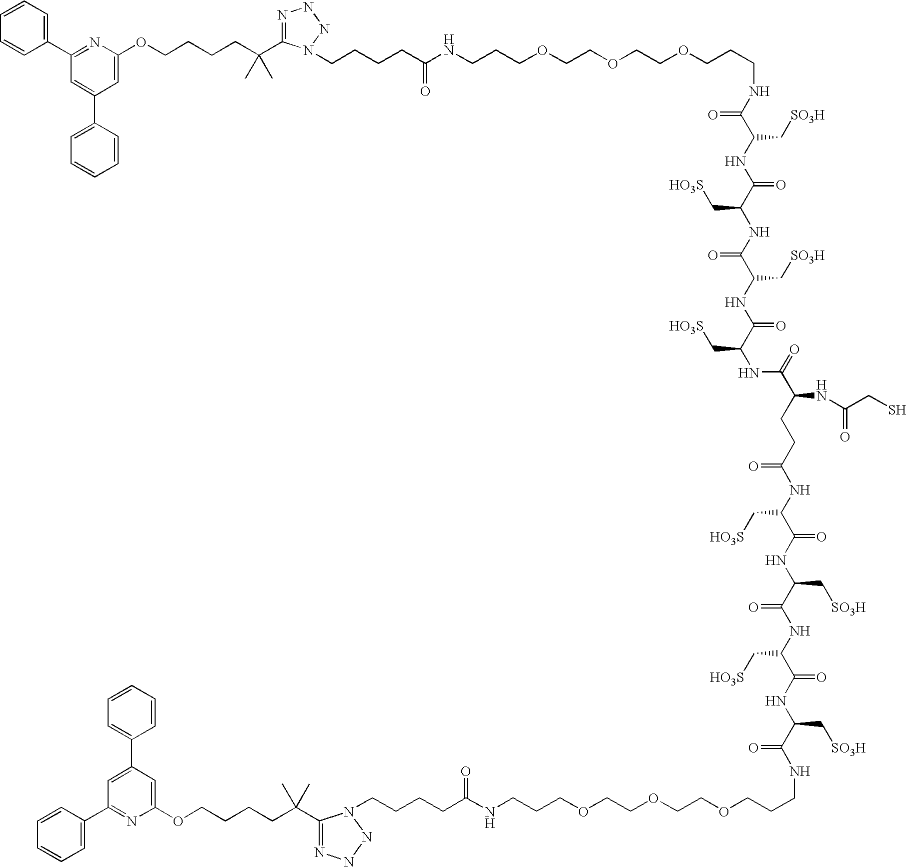

### Part A—Preparation of (2R,5R,8R,11R,14S,19R,22R,25R,28R)-2,28-bis(19-(5-(6-(4,6-Diphenylpyridin-2-yloxy)-2-methylhexan-2-yl)-1H-tetrazol-1-yl)-15-oxo-4,7,10-trioxa-14-azanonadecylcarbamoyl)-4,7,10,13,17,20,23,26-octaoxo-5,8,11,19,22,25-hexakis(sulfomethyl)-14-(2-(tritylthio)acetamido)-3,6,9,12,18,21,24,27-octaazanonacosane-1,29-disulfonic Acid

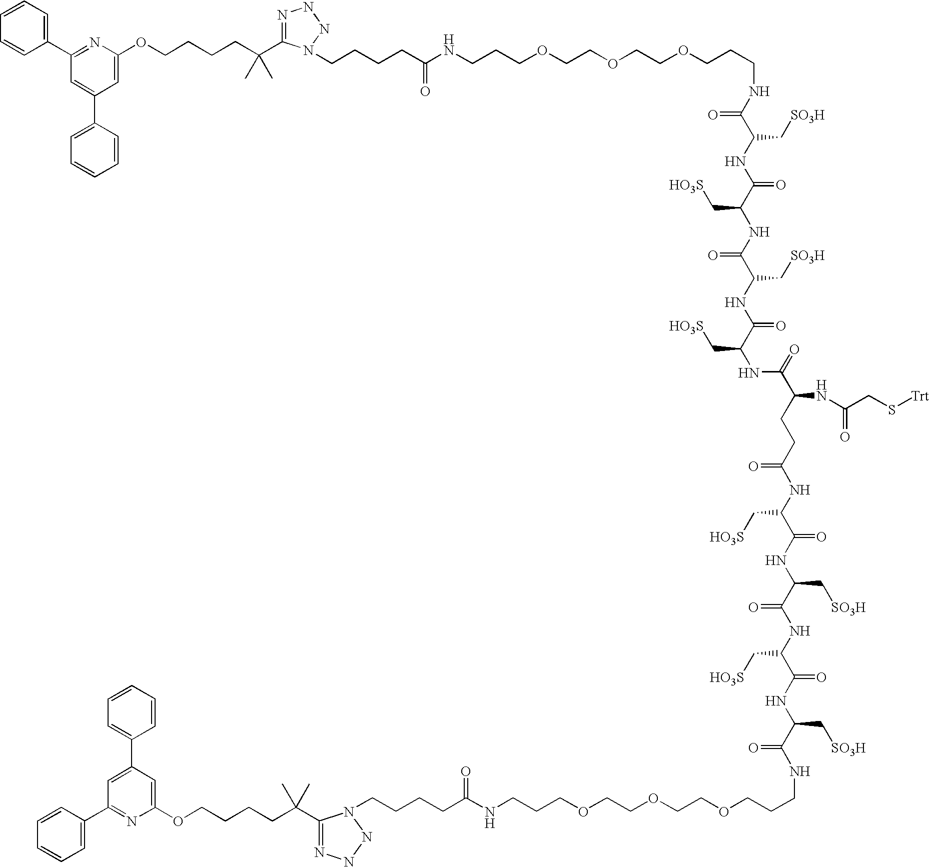

A solution of 2-((triphenylmethyl)thio)acetic acid and HOAt in DMF is pre-activated by the addition of HBTU and sufficient DIEA to maintain pH 8-9. To the solution is added (2R,5R,8R,11R,14S,19R,22R,25R,28R)-14-amino-2,28-bis(19-(5-(6-(4,6-diphenylpyridin-2-yloxy)-2-methylhexan-2-yl)-1H-tetrazol-1-yl)-15-oxo-4,7,10-trioxa-14-azanonadecylcarbamoyl)-4,7,10,13,17,20,23,26-octaoxo-5,8,11,19,22,25-hexakis(sulfomethyl)-3,6,9,12,18,21,24,27-octaazanonacosane-1,29-disulfonic acid (Harris, T. D., et al. J. Med. Chem. 2005, 48, 6442-6253), and the solution is stirred at room temperature under nitrogen for 18 h. DMF is removed in vacuo and the crude product is purified by preparative reverse phase HPLC to give the title compound.

### Part B—Preparation of (2R,5R,8R,11R,14S,19R,22R,25R,28R)-2,28-bis(19-(5-(6-(4,6-Diphenylpyridin-2-yloxy)-2-methylhexan-2-yl)-1H-tetrazol-1-yl)-15-oxo-4,7,10-trioxa-14-azanonadecylcarbamoyl)-14-(2-mercaptoacetamido)-4,7,10,13,17,20,23,26-octaoxo-5,8,11,19,22,25-hexakis(sulfomethyl)-3,6,9,12,18,21,24,27-octaazanonacosane-1,29-disulfonic Acid

A solution of the product of Part A in TFA:triethylsilane (95:5) is heated at 70° C. under nitrogen for 1 h. The solution is concentrated on a rotary evaporator and dried further under high vacuum. The resulting residue is purified by reverse phase HPLC to give the title compound.

### EXAMPLE 3

### Synthesis of LTB₄ Receptor-Targeted-Paramagnetic Nanoparticles Using a Monomeric LTB4 Antagonist

### Part A—Preparation of a Monomeric LTB₄ Receptor-Targeting-Surfactant Conjugate

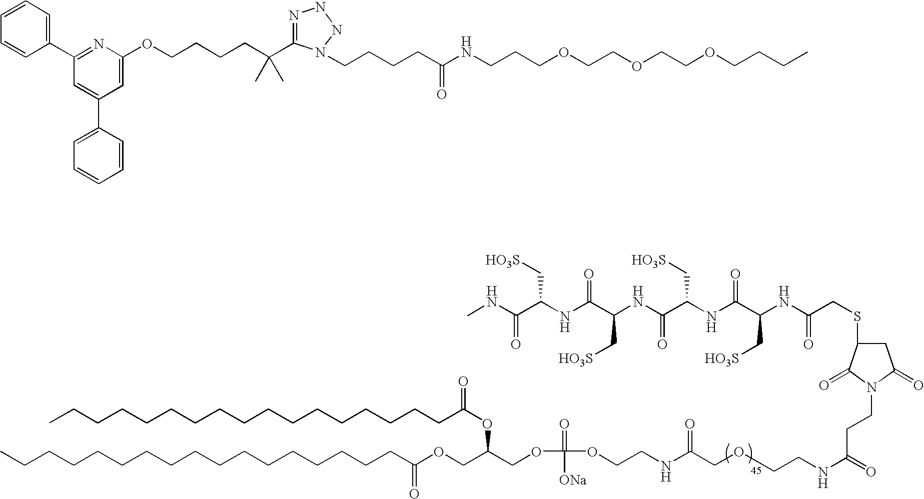

1,2-Distearoyl-sn-Glycero-3-Phosphoethanolamine-N-[Maleimide(Polyethylene Glycol)2000] is dissolved in DMF and degassed by sparging with nitrogen or argon. The oxygen-free solution is adjusted to pH 7-8 using DIEA, and treated with the product of Example 1, Part B. Stirring is continued at ambient temperatures until analysis indicates complete consumption of starting materials. DMF is removed in vacuo and the crude product is purified by preparative HPLC to obtain the title compound.

### Part B—Preparation of LTB₄ Receptor-Targeted Paramagnetic Nanoparticles

The paramagnetic nanoparticles are produced as described in Flacke, S., et al., Circulation 2001, 104, 1280-1285. Briefly, a mixture of 40% (v/v) perfluorooctylbromide (PFOB), 2% (w/v) of a surfactant co-mixture as described below, 1.7% (w/v) glycerin, and water representing the balance, is emulsified in a M110S Microfluidics emulsifier (Microfluidics, Newton, Mass.) at 20,000 PSI for four minutes. The completed emulsion is placed in crimp-sealed vials under a nitrogen head space. The surfactant co-mixture consists of 60 mole % lecithin, 0.05 mole % the product of Part A, 8 mole % cholesterol, 30 mole % gadolinium diethylenetriaminepentaacetic acid-bisoleate (Gd-DTPA-BOA, Gateway Chemical Technologies, St. Louis, Mo.) and 1.95 mole % dipalmitoyl-phosphatidylethanolamine (DPPE) (Avanti Polar Lipids, Inc., Alabaster, Ala.).

### EXAMPLE 4

### Preparation of LTB₄ Receptor-Targeted Nonparamagnetic Nanoparticles

LTB₄-targeted nonparamagnetic control nanoparticles are prepared in an identical fashion to the targeted formulation of Example 3, except that the lipophilic Gd³⁺ chelate is replaced in the surfactant co-mixture with increased lecithin (70 mole %) and cholesterol (28 mole %).

### EXAMPLE 5

### Preparation of Nontargeted Paramagnetic Nanoparticles

Nontargeted paramagnetic control nanoparticles are prepared in an identical fashion to the targeted formulation of Example 3, except that the LTB₄ antagonist-surfactant conjugate is replaced in the surfactant co-mixture with increased DPPE (2 mole %).

### EXAMPLE 6

### Synthesis of LTB₄ Receptor-Targeted-Paramagnetic Nanoparticles Using a Dimeric LTB4 Antagonist

### Part A—Preparation of a Dimeric LTB₄ Receptor-Targeting-Surfactant Conjugate

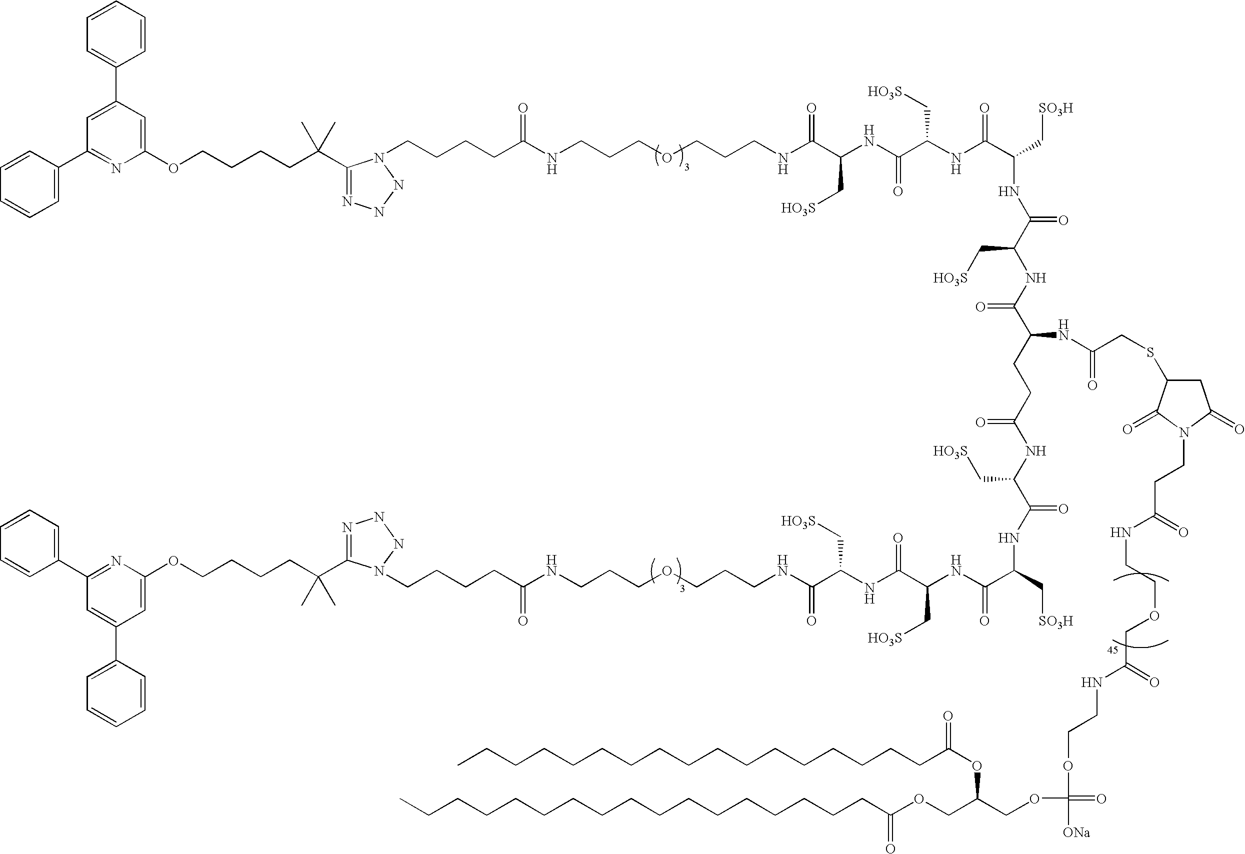

1,2-Distearoyl-sn-Glycero-3-Phosphoethanolamine-N-[Maleimide(Polyethylene Glycol)2000] is dissolved in DMF and degassed by sparging with nitrogen or argon. The oxygen-free solution is adjusted to pH 7-8 using DIEA, and treated with the product of Example 2, Part B. Stirring is continued at ambient temperatures until analysis indicates complete consumption of starting materials. DMF is removed in vacuo and the crude product is purified by preparative HPLC to obtain the title compound.

### Part B—Preparation of LTB₄ Receptor-Targeted Paramagnetic Nanoparticles

The receptor-targeted-paramagnetic nanoparticles having a dimeric LTB4 antagonist are prepared according to the procedure of Example 3, Part B by substituting the product of Part A, above.

Various modifications of the invention, in addition to those described herein, will be apparent to those skilled in the art from the foregoing description. Such modifications are also intended to fall within the scope of the appended claims.

What is claimed is:

1. A method to deliver an emulsion comprising nanoparticles to a target tissue, wherein said target tissue is characterized as a LTB4 receptor, which method comprises administering to a subject comprising such tissue an emulsion of nanoparticles wherein said nanoparticles are coupled to a ligand specific for LTB4 receptors, with the proviso that said ligand is other than an antibody or fragment thereof.

2. The method of claim 1, wherein said ligand is represented by the compound of the formula: 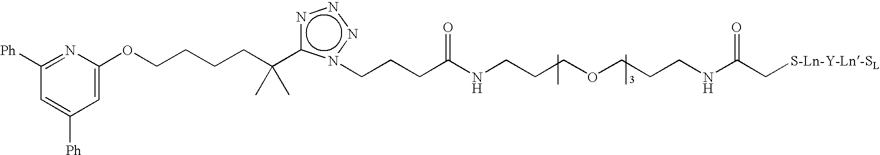
wherein Ln, Ln′, Y and S_{L} are defined as follows, wherein Lₙ is a linking group having the formula:
(CR⁸R⁹)_{g}—(W¹)ₕ-(M¹)ₖ-(CR¹⁰R¹¹)_{g}
wherein,
R⁸, R⁹, R¹⁰ and R¹¹ are independently selected at each occurrence from the group: a bond to L_{n′}, H, and C₁-C₃ alkyl or R¹⁰ and R¹¹ may be taken together to form a 3-6 membered cycloalkyl or heterocycle;
W¹ is O;
M¹ is selected from the group of: phenyl substituted with 0-1 R¹², heterocycle substituted with 0-1 R¹², benzophenone substituted with 0-1 R¹², and diphenylether substituted with 0-1 R¹²;
R¹² is independently selected from the group: a bond to L_{n′}, —COOR¹³, C₁-C₅ alkyl substituted with 0-1 R¹⁴, and C₁-C₅ alkoxy substituted with 0-1 R¹⁴;
R¹³ is H or C₁-C₅ alkyl;
R¹⁴ is independently selected from the group: a bond to L_{n′}, and —COOH;
g is 0-10;
h is 0-3;
k is 0-1;
g is 0-5; provided that when h is 0 and k is 0, g is >1; and provided that when W¹ is O or S and k is 0, g+g′ is ≧1;
Y is selected from C(═O)NH, NHC(═O), C═O, C(═O)O, OC(═O), NHS(═O)₂, C(═O)NHS(═O)₂, COOH, C(═O)NH₂, NH(C═O)NH, S, or tetrazole;
provided that from 0-1 of R⁹, R¹⁰, R¹¹, R¹², and R¹⁴ is a bond to L_{n′} and when one of these variables is a bond to L_{n′} then Y is COOH, C(═O)NH₂, or tetrazole;
L_{n′} is a linking group having the formula:
(W²)_{h′}—(CR¹⁵R¹⁶)_{g″}-(M²)_{k′}-(W²)_{h″}—(CR¹⁷R¹⁸)_{g′″}—(W²)_{h′″}
wherein, W² is independently selected at each occurrence from the group: O, S, NH, NHC(═O), C(═O)NH, C(═O), C(═O)O, OC(═O), NHC(═O)NH, SO₂, (OCH₂CH₂)ₛ, (CH₂CH₂O)_{s′}, (OCH₂CH₂CH₂)_{s″}, (CH₂CH₂CH₂O)ₜ, CH₂S, SCH₂, and (aa)_{t′}, wherein aa is independently at each occurrence an amino acid, and s, s′, s″, t, and t′ are independently 1-10;
M² is selected from the group of: aryl substituted with 0-1 R¹⁹, cycloalkyl substituted with 0-3 R¹⁹, and heterocycle substituted with 0-1 R¹⁹;
R¹⁵, R¹⁶, R¹⁷ and R¹⁸ are independently selected at each occurrence from the group: ═O, COOH, SO₃H, PO₃H, C₁-C₅ alkyl substituted with 0-3 K¹⁹, aryl substituted with 0-3 R¹⁹, benzyl substituted with 0-3 R¹⁹, and C₁-C₅ alkoxy substituted with 0-3 R¹⁹, NHC(═O)R²⁰, C(═O)NHR²⁰, NHC(═O)NHR²⁰, NHR²⁰, R²⁰, and a bond to a lipid/surfactant;
Optionally, R¹⁷ and R¹⁸ may form a 4-7 membered heterocyclic or aliphatic ring; R¹⁹ is independently selected at each occurrence from the group of: COOR²⁰, OH, NHR²⁰, SO₃H, PO₃H, aryl substituted with 0-3 R²⁰, heterocycle substituted with 0-3 R²⁰, C₁-C₅ alkyl substituted with 0-1 R²¹, C₁-C₅ alkoxy substituted with 0-1 R²¹, a bond, and a bond to a lipid/surfactant;
R²⁰ is independently selected at each occurrence from the group of: H, aryl substituted with 0-1 R²¹, heterocycle substituted with 0-1 R²¹, cycloalkyl substituted with 0-1 R²¹, polyalkylene glycol substituted with 0-1 R²¹, carbohydrate substituted with 0-1 R²¹, cyclodextrin substituted with 0-1 R2¹, amino acid substituted with 0-1 R²¹, polycarboxyalkyl substituted with 0-1 R²¹, polyazaalkyl substituted with 0-1 R²¹, peptide substituted with 0-1 R²¹, wherein said peptide is comprised of 2-10 amino acids, and a bond to a lipid/surfactant;
R²¹ is a bond to a lipid/surfactant;
and k′ is 0-2; h′ is 0-2; h″ is 0-5; h′″ is 0-2; g″ is 0-10; g′″ is 0-10; and
S_{L} is a phospholipid or a cationic lipid selected from the group consisting of DOTMA, N-[1 -(2,3-dioleoyloxy)propyl]-N,N,N-trimethylammonium chloride; DOTAP, 1,2-dioleoyloxy-3-(trimethylammon-io)propane; DOTB, 1,2-dioleoyl-3-(4′-trimethylammonio)butanoyl-sn-glycerol-, 1,2-diacyl-3-trimethylammonium-propane; 1,2-diacyl-3-dimethylammonium-pr-opane; 1,2-diacyl-sn-glycerol-3-ethyl phosphocholine; and 3.beta.-[N′,N′-dimethylaminoethane)-carbamol]cholesterol-HCl.

3. The method of claim 2, wherein S_{L} is a phospholipid selected from the group consisting of dialkylphosphatidyl ethanolamine, dialkylphosphatidyl choline, dialkylphosphatidyl serine, DPPA, DPPE, and DPPC.

4. A method of diagnosing and locating inflammation which comprises administering to a subject comprising such tissue an emulsion of nanoparticles wherein said nanoparticles are coupled to a ligand specific for LTB4 receptors, with the proviso that said ligand is other than an antibody or fragment thereof.

5. The method of claim 4, wherein said ligand is represented by the compound of the formula: 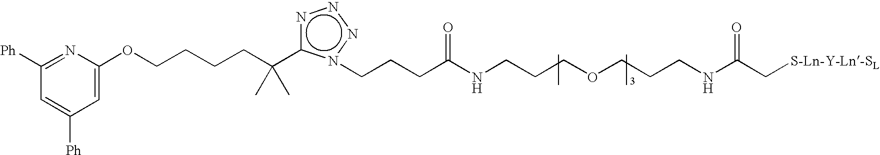
wherein Ln, Ln′, Y and S_{L} are as set forth as follows, wherein Lₙ is a linking group having the formula:
(CR⁸R⁹)_{g}—(W¹)ₕ-(M¹)ₖ-(CR¹⁰R¹¹)_{g}
wherein,
R⁸, R⁹, R¹⁰ and R¹¹ are independently selected at each occurrence from the group: a bond to L_{n′}, H, and C₁-C₃ alkyl or R¹⁰ and R¹¹ may be taken together to form a 3-6 membered cycloalkyl or heterocycle;
W¹ is O;
M¹ is selected from the group of: phenyl substituted with 0-1 R¹², heterocycle substituted with 0-1 R¹², benzophenone substituted with 0-1 R¹², and diphenylether substituted with 0-1 R¹²;
R¹² is independently selected from the group: a bond to L_{n′}, —COOR^{13,} C₁-C₅ alkyl substituted with 0-1 R¹⁴, and C₁-C₅ alkoxy substituted with 0-1 R¹⁴;
R¹³ is H or C₁-C₅ alkyl;
R¹⁴ is independently selected from the group: a bond to L_{n′}, and —COOH;
g is 0-10;
h is 0-3;
k is 0-1;
g is 0-5; provided that when h is 0 and k is 0, g is >1; and provided that when W¹ is O or S and k is 0, g+g′ is ≧1;
Y is selected from C(═O)NH, NHC(═O), C═O, C(═O)O, OC(═O), NHS(═O)₂, C(═O)NHS(═O)₂, COOH, C(═O)NH₂, NH(C═O)NH, S, or tetrazole;
provided that from 0-1 of R⁹, R¹⁰, R¹¹, R¹², and R¹⁴ is a bond to L_{n′} and when one of these variables is a bond to L_{n′} then Y is COOH, C(═O)NH₂, or tetrazole;
L_{n′} is a linking group having the formula:
(W²)_{h′}—(CR¹⁵R¹⁶)_{g″}-(M²)_{k′}-(W²)_{h″}—(CR¹⁷R¹⁹)_{g}′″—(W²)_{h′″}
wherein, W² is independently selected at each occurrence from the group: O, S, NH, NHC(═O), C(═O)NH, C(═O), C(═O)O, OC(═O), NHC(═O)NH, SO₂, (OCH₂CH₂)ₛ, (CH₂CH₂O)_{s′}, (OCH₂CH₂CH₂)_{s″}, (CH₂CH₂CH₂O)ₜ, CH₂S, SCH₂, and (aa)_{t′}, wherein aa is independently at each occurrence an amino acid, and s, s′, s″, t, and t′ are independently 1-10;
M² is selected from the group of: aryl substituted with 0-1 R¹⁹, cycloalkyl substituted with 0-3 R¹⁹, and heterocycle substituted with 0-1 R¹⁹;
R¹⁵, R¹⁶, R¹⁷ and R¹⁸ are independently selected at each occurrence from the group: ═O, COOH, SO₃H, PO₃H, C₁-C₅ alkyl substituted with 0-3 R¹⁹, aryl substituted with 0-3 R¹⁹, benzyl substituted with 0-3 R¹⁹, and C₁-C₅ alkoxy substituted with 0-3 R¹⁹, NHC(═O)R²⁰, C(═O)NHR²⁰, NHC(=O)NHR²⁰, NHR²⁰, R²⁰ and a bond to a lipid/surfactant;
Optionally, R¹⁷ and R¹⁸ may form a 4-7 membered heterocyclic or aliphatic ring;
R¹⁹ is independently selected at each occurrence from the group of: COOR²⁰, OH, NHR²⁰, SO₃H, PO₃H, aryl substituted with 0-3 R²⁰, heterocycle substituted with 0-3 R²⁰, C₁-C₅ alkyl substituted with 0-1 R²¹, C₁-C₅ alkoxy substituted with 0-1 R²¹, a bond, and a bond to a lipid/surfactant;
R²⁰ is independently selected at each occurrence from the group of: H, aryl substituted with 0-1 R²¹, heterocycle substituted with 0-1 R²¹, cycloalkyl substituted with 0-1 R²¹, polyalkylene glycol substituted with 0-1 R²¹, carbohydrate substituted with 0-1 R²¹, cyclodextrin substituted with 0-1 R2¹, amino acid substituted with 0-1 R²¹, polycarboxyalkyl substituted with 0-1 R²¹, polyazaalkyl substituted with 0-1 R²¹, peptide substituted with 0-1 R²¹, wherein said peptide is comprised of 2-10 amino acids, and a bond to a lipid/surfactant;
R²¹ is a bond to a lipid/surfactant;
and k′ is 0-2; h′ is 0-2; h″ is 0-5; h′″ is 0-2; g″ is 0-10; g′″ is 0-10; and
S_{L} is a phospholipid.

6. The method of claim 5, wherein S_{L} is a phospholipid selected from the group consisting of dialkylphosphatidyl ethanolamine, dialkylphosphatidyl choline, dialkylphosphatidyl serine, DPPA, DPPE, and DPPC.

7. A composition comprising an emulsion of nanoparticles, wherein said nanoparticles are coupled to a ligand specific for LTB4 receptors, with the proviso that said ligand is other than an antibody or fragment thereof.

8. The composition of claim 7, wherein said ligand is represented by the compound of the formula: 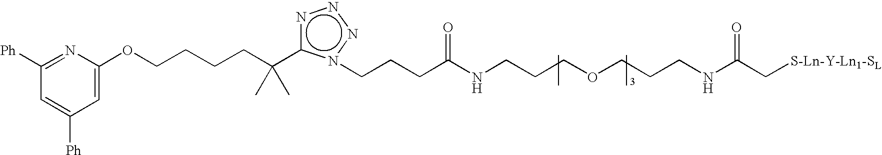
wherein Ln, Ln′, Y and S_{L} are as set forth as follows, wherein Lₙ is a linking group having the formula:
(CR⁸R⁹)_{g}—(W¹)ₕ-(M¹)ₖ-(CR¹⁰R¹¹)_{g}
wherein,
R⁸, R⁹, R¹⁰ and R¹¹ are independently selected at each occurrence from the group: a bond to L_{n′}, H, and C₁-C₃ alkyl or R¹⁰ and R¹¹ may be taken together to form a 3-6 membered cycloalkyl or heterocycle;
W¹ is O;
M¹ is selected from the group of: phenyl substituted with 0-1 R¹², heterocycle substituted with 0-1 R¹², benzophenone substituted with 0-1 R¹², and diphenylether substituted with 0-1 R¹²;
R¹² is independently selected from the group: a bond to L_{n′}, —COOR^{13,} C₁-C₅ alkyl substituted with 0-1 R¹⁴, and C₁-C₅ alkoxy substituted with 0-1 R¹⁴;
R¹³ is H or C₁-C₅ alkyl:
R¹⁴ is independently selected from the group: a bond to L_{n′}, and —COOH;
g is 0-10;
h is 0-3;
k is 0-1;
g is 0-5; provided that when h is 0 and k is 0, g is >1; and provided that when W¹ is O or S and k is 0, g+g′ is ≧1;
Y is selected from C(═O)NH, NHC(═O), C═O, C(═O)O, OC(═O), NHS(═O)₂, C(═O)NHS(═O)₂, COOH, C(═O)NH₂, NH(C═O)NH, S, or tetrazole;
provided that from 0-1 of R⁹, R¹⁰, R¹¹, R¹², and R¹⁴ is a bond to L_{n′} and when one of these variables is a bond to L_{n′} then Y is COOH, C(═O)NH₂, or tetrazole;
L_{n′} is a linking group having the formula:
(W²)_{h′}—(CR¹⁵R¹⁶)_{g″}-(M²)_{k′}-(W²)_{h″}—(CR¹⁷R¹⁸)_{g′″}—(W²)_{h′″}
wherein, W² is independently selected at each occurrence from the group: O, S, NH, NHC(═O), C(═O)NH, C(═O), C(═O)O, OC(═O), NHC(═O)NH, SO₂, (OCH₂CH₂)ₛ, (CH₂CH₂O)_{s′}, (OCH₂CH₂CH₂)_{s″}, (CH₂CH₂CH₂O)ₜ, CH₂S, SCH₂, and (aa)_{t′}, wherein aa is independently at each occurrence an amino acid, and s, s′, s″, t, and t′ are independently 1-10;
M² is selected from the group of: aryl substituted with 0-1 R¹⁹, cycloalkyl substituted with 0-3 R¹⁹, and heterocycle substituted with 0-1 R¹⁹;
R¹⁵, R¹⁶, R¹⁷ and R¹⁸ are independently selected at each occurrence from the group: ═O, COOH, SO₃H, PO₃H, C₁-C₅ alkyl substituted with 0-3 R¹⁹, aryl substituted with 0-3 R¹⁹, benzyl substituted with 0-3 R¹⁹, and C₁-C₅ alkoxy substituted with 0-3 R¹⁹, NHC(═O)R²⁰, C(═O)NHR²⁰, NHC(═O)NHR²⁰, NHR²⁰, R²⁰, and a bond to a lipid/surfactant;
Optionally, R¹⁷ and R¹⁸ may form a 4-7 membered heterocyclic or aliphatic ring;
R¹⁹ is independently selected at each occurrence from the group of: COOR²⁰, OH, NHR²⁰, SO₃H, PO₃H, aryl substituted with 0-3 R²⁰, heterocycle substituted with 0-3 R²⁰, C₁-C₅ alkyl substituted with 0-1 R₂₁, C₁-C₅ alkoxy substituted with 0-1 R²¹, a bond, and a bond to a lipid/surfactant;
R²⁰ is independently selected at each occurrence from the group of: H, aryl substituted with 0-1 R²¹, heterocycle substituted with 0-1 R²¹, cycloalkyl substituted with 0-1 R²¹, polyalkylene glycol substituted with 0-1 R²¹, carbohydrate substituted with 0-1 R²¹, cyclodextrin substituted with 0-1 R²¹, amino acid substituted with 0-1 R²¹, polycarboxyalkyl substituted with 0-1 R²¹, polyazaalkyl substituted with 0-1 R²¹, peptide substituted with 0-1 R²¹, wherein said peptide is comprised of 2-10 amino acids, and a bond to a lipid/surfactant;
R²¹ is a bond to a lipid/surfactant;
and k′ is 0-2; h′ is 0-2; h″ is 0-5; h′″ is 0-2; g″ is 0-10; g′″ is 0-10; and
S_{L} is a phospholipid or a cationic lipid selected from the group consisting of DOTMA, N-[1-(2,3-dioleoyloxy)propyl]-N,N,N-trimethylammonium chloride; DOTAP, 1,2-dioleoyloxy-3-(trimethylammon-io)propane; DOTB, 1,2-dioleoyl-3-(4′-trimethylammonio)butanoyl-sn-glycerol-, 1,2-diacyl-3-trimethylammonium-propane; 1,2-diacyl-3-dimethylammonium-pr-opane; 1,2-diacyl-sn-glycerol-3-ethyl phosphocholine; and 3.beta.-[N′,N′-dimethylaminoethane)-carbamol]cholesterol-HCl.

9. The composition of claim 8, wherein S_{L} is a phospholipid selected from the group consisting of dialkylphosphatidyl ethanolamine, dialkylphosphatidyl choline, dialkylphosphatidyl serine, DPPA, DPPE, and DPPC.

10. A kit for the preparation of an emulsion of nanoparticles targeted to tissue having an LTB4 receptor, which kit comprises at least one container that contains nanoparticles comprising a ligand specific for LTB4 receptors and a linking moiety for coupling to an ancillary agent.

11. A kit for the preparation of an emulsion of nanoparticles targeted to tissue having an LTB4 receptor, which kit comprises at least one container that contains nanoparticles comprising a linking moiety for coupling to a ligand specific for LTB4 receptors and at least one container that contains a ligand specific for LTB4 receptors.

12. A compound of the formula:

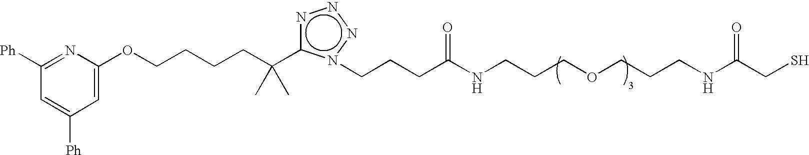

13. The compound of claim 12, wherein said compound is conjugated to one or more nanoparticles.

14. The compound of claim 13, wherein said compound is part of an emulsion.

15. A compound of the formula: 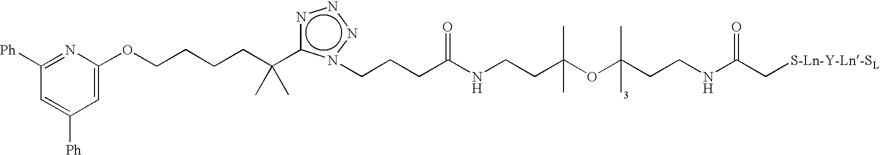
wherein Lₙ, L_{n′} and Y are as set forth as follows, wherein Lₙ is a linking group having the formula:
(CR⁸R⁹)_{g}—(W¹)ₕ-(M¹)ₖ-(CR¹⁰R¹¹)_{g}
wherein,
R⁸, R⁹, R¹⁰ and R¹¹ are independently selected at each occurrence from the group: a bond to L_{n′}, H, and C₁-C₃ alkyl or R¹⁰ and R¹¹ may be taken together to form a 3-6 membered cycloalkyl or heterocycle;
W¹ is O;
M¹ is selected from the group of: phenyl substituted with 0-1 R¹², heterocycle substituted with 0-1 R¹², benzophenone substituted with 0-1 R¹², and diphenylether substituted with 0-1 R¹²;
R¹² is independently selected from the group: a bond to L_{n′}, —COOR¹³, C₁-C₅ alkyl substituted with 0-1 R¹⁴, and C₁-C₅ alkoxy substituted with 0-1 R¹⁴;
R¹³ is H or C₁-C₅ alkyl:
R¹⁴ is independently selected from the group: a bond to L_{n′}, and —COOH;
g is 0-10;
h is 0-3;
k is 0-1;
g is 0-5; provided that when h is 0 and k is 0, g is >1; and provided that when W¹ is O or S and k is 0, g+g′ is ≧1;
Y is selected from C(═O)NH, NHC(═O), C═O, C(═O)O, OC(═O), NHS(═O)₂, C(═O)NHS(═O)₂, COOH, C(═O)NH₂, NH(C═O)NH, S, or tetrazole;
provided that from 0-1 of R⁹, R¹⁰, R¹¹, R¹², and R¹⁴ is a bond to L_{n′} and when one of these variables is a bond to L_{n′} then Y is COOH, C(═O)NH₂, or tetrazole;
L_{n′} is a linking group having the formula:
(W²)_{h′}—(CR¹⁵R¹⁶)_{g″}-(M²)_{k′}-(W²)_{h″}—(CR¹⁷R¹⁸)_{g′″}—(W²)_{h′″}
wherein, W² is independently selected at each occurrence from the group: O, S, NH, NHC(═O), C(═O)NH, C(═O), C(═O)O, OC(═O), NHC(═O)NH, SO₂, (OCH₂CH₂)ₛ, (CH₂CH₂O)_{s′}, (OCH₂CH₂CH₂)_{s″}, (CH₂CH₂CH₂O)ₜ, CH₂S, SCH₂, and (aa)_{t′}, wherein aa is independently at each occurrence an amino acid, and s, s′, s″, t, and t′ are independently 1-10;
M² is selected from the group of: aryl substituted with 0-1 R¹⁹, cycloalkyl substituted with 0-3 R¹⁹, and heterocycle substituted with 0-1 R¹⁹;
R¹⁵, R¹⁶, R¹⁷ and R¹⁸ are independently selected at each occurrence from the group: ═O, COOH, SO₃H, PO₃H, C₁-C₅ alkyl substituted with 0-3 R¹⁹, aryl substituted with 0-3 R¹⁹, benzyl substituted with 0-3 R¹⁹, and C₁-C₅ alkoxy substituted with 0-3 R¹⁹, NHC(═O)R²⁰, C(═O)NHR²⁰, NHC(═O)NHR²⁰, NHR²⁰, R²⁰ and a bond to a lipid/surfactant;
Optionally, R¹⁷ and R¹⁸ may form a 4-7 membered heterocyclic or aliphatic ring;
R¹⁹ is independently selected at each occurrence from the group of: COOR²⁰, OH, NHR²⁰, SO₃H, PO₃H, aryl substituted with 0-3 R²⁰, heterocycle substituted with 0-3 R²⁰, C₁-C₅ alkyl substituted with 0-1 R²¹, C₁-C₅ alkoxy substituted with 0-1 R²¹, a bond, and a bond to a lipid/surfactant;
R²⁰ is independently selected at each occurrence from the group of: H, aryl substituted with 0-1 R²¹, heterocycle substituted with 0-1 R²¹, cycloalkyl substituted with 0-1 R²¹, polyalkylene glycol substituted with 0-1 R²¹, carbohydrate substituted with 0-1 R²¹, cyclodextrin substituted with 0-1 R2¹, amino acid substituted with 0-1 R²¹, polycarboxyalkyl substituted with 0-1 R²¹, polyazaalkyl substituted with 0-1 R²¹, peptide substituted with 0-1 R²¹, wherein said peptide is comprised of 2-10 amino acids, and a bond to a lipid/surfactant;
R²¹ is a bond to a lipid/surfactant;
and k′ is 0-2; h′ is 0-2; h″ is 0-5; h′″ is 0-2; g″ is 0-10; g′″ is 0-10;
and S_{L} is a phospholipid or a cationic lipid selected from the group consisting of DOTMA, N-[1-(2,3 -dioleoyloxy)propyl]-N,N,N-trimethylammonium chloride;
DOTAP, 1,2-dioleoyloxy-3-(trimethylammon-io)propane; DOTB, 1,2-dioleoyl-3-(4′-trimethylammonio)butanoyl-sn-glycerol-, 1,2-diacyl-3-trimethylammonium-propane; 1,2-diacyl-3-dimethylammonium-pr-opane; 1,2-diacyl-sn-glycerol-3-ethyl phosphocholine; and 3.beta.-[N′,N′-dimethylaminoethane)-carbamol]cholesterol-HCl.

16. The compound of claim 15, wherein said compound is conjugated to one or more nanoparticles.

17. The compound of claim 16, wherein said compound is part of an emulsion.

18. A compound having the following formula:

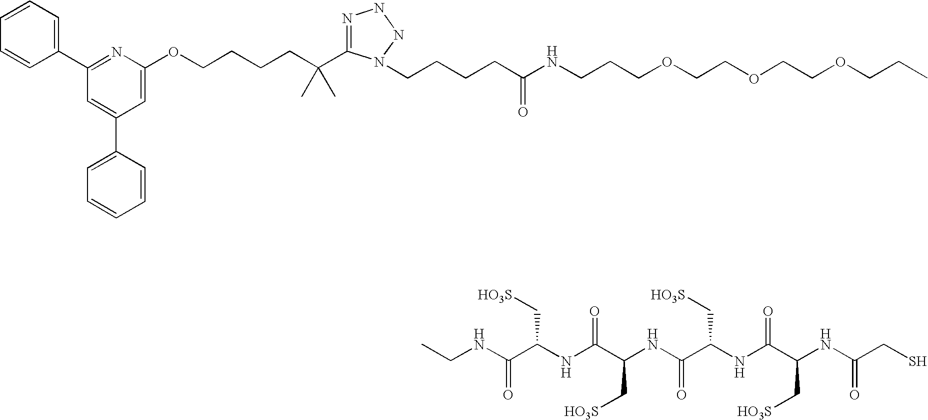

19. A compound having the following formula:

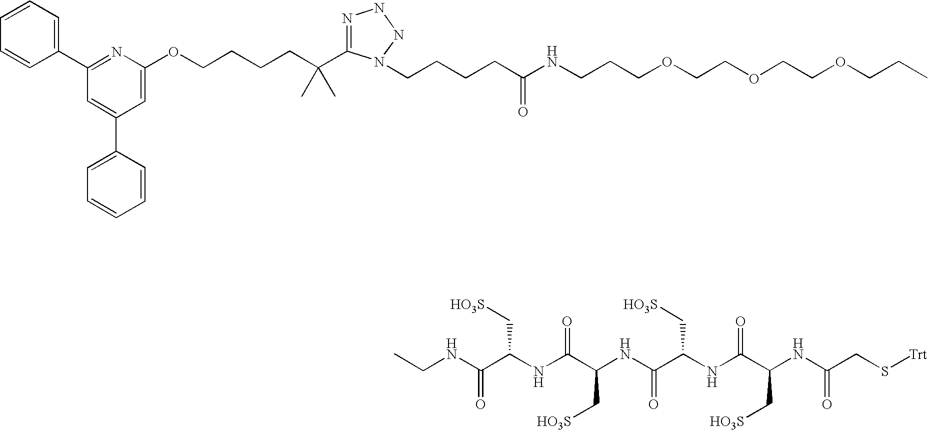

20. A compound having the following formula:

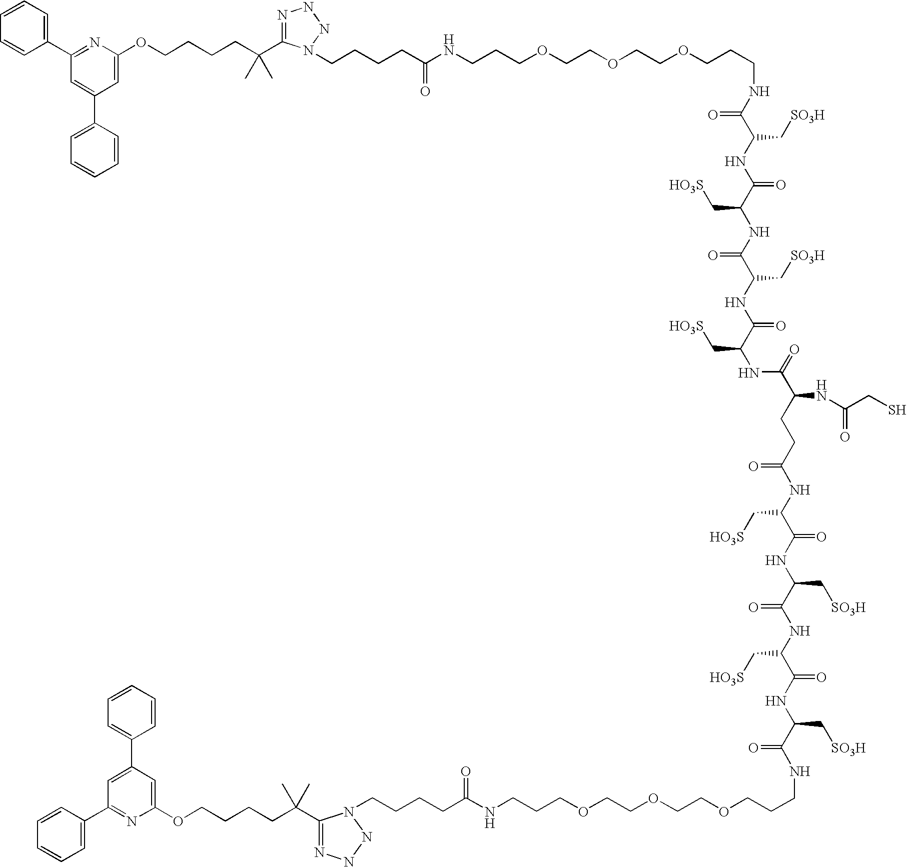

21. A compound having the following formula:

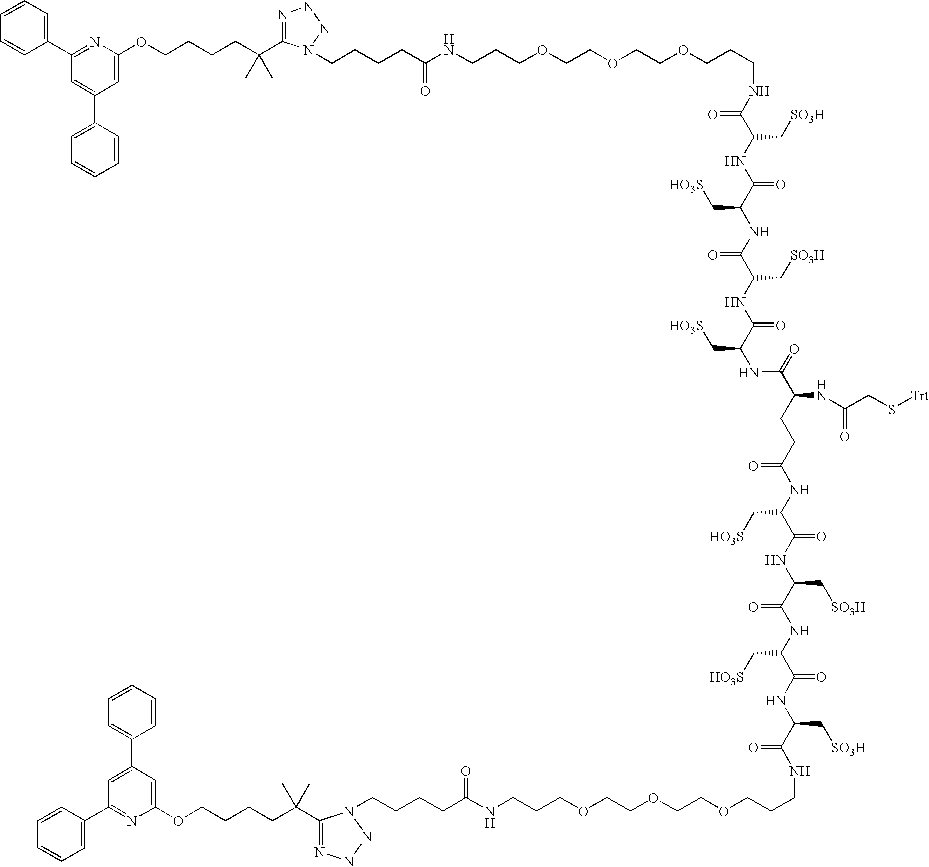

22. A compound having the following formula:

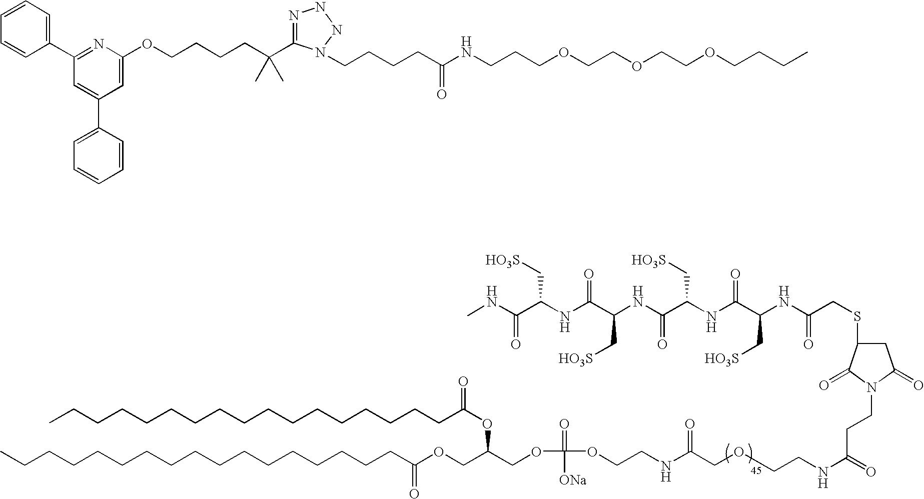

23. A compound having the following formula:

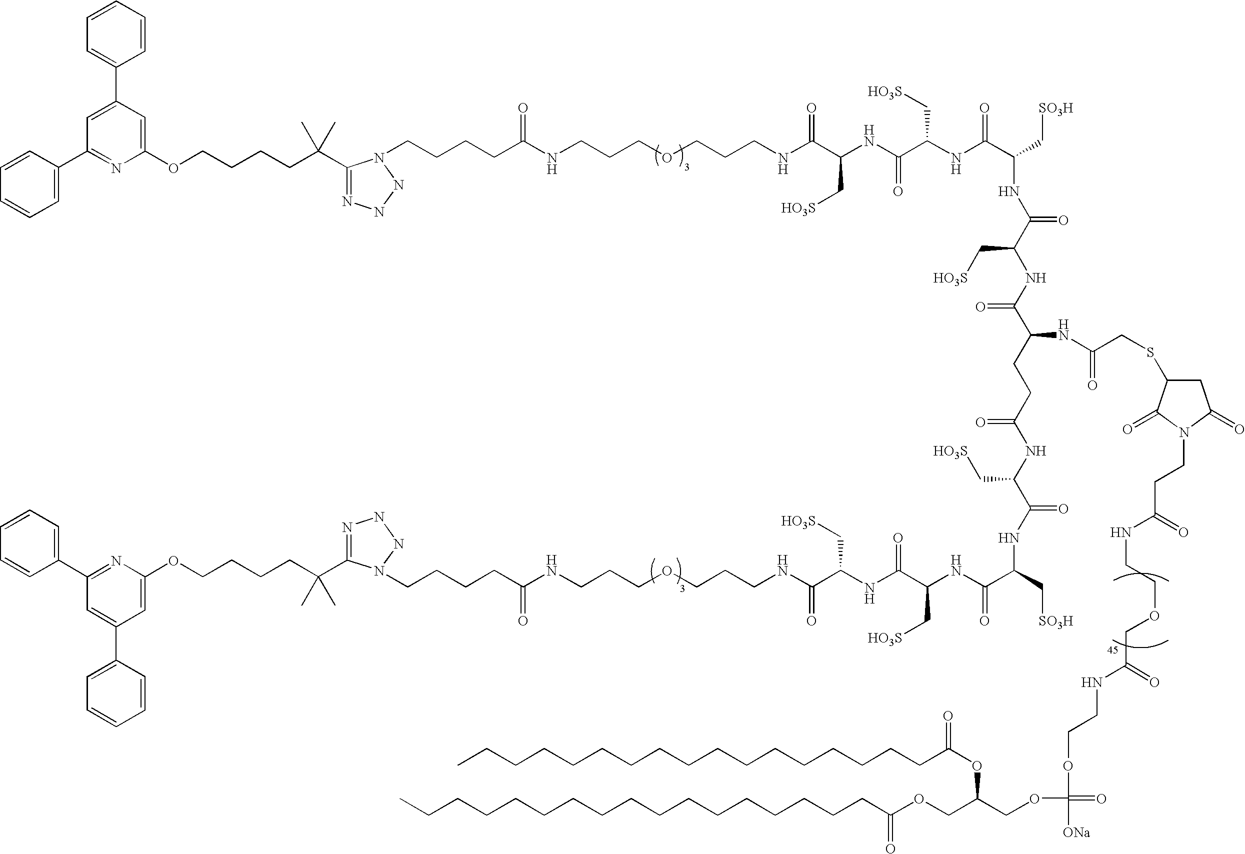

24. A compound having the following formula: [chemical structure]
